(19) United States
(12) Patent Application Publication
(10) Pub. No.: US 2005/0176600 A1
(43) Pub. Date: Aug. 11, 2005

(54) Detergent cosmetic compositions comprising at least one silicone and at least one drawing polymer, and use thereof

(76) Inventors: Henri Samain, Bievres (FR); Geraldine Fack, Levallois (FR); Franck Giroud, Clichy (FR); Laurence Paul, Saint Leu La Foret (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/030,308

(22) Filed: Jan. 7, 2005

Related U.S. Application Data
(60) Provisional application No. 60/537,898, filed on Jan. 22, 2004.
(60) Provisional application No. 60/538,248, filed on Jan. 23, 2004.

(30) Foreign Application Priority Data
Jan. 7, 2004 (FR) 04 50031
Jan. 7, 2004 (FR) 04 50039

(51) Int. Cl. A61K 7/075

(52) U.S. Cl. 510/119

(57) ABSTRACT

Disclosed herein are novel washing compositions, for example hair compositions, comprising, in a cosmetically acceptable medium, at least one nonaminated silicone with a viscosity lower than 100,000 cSt, at least one anionic, nonionic, or amphoteric detergent surfactant, and at least one drawing polymer. These compositions may have an improved styling effect.

This application claims benefit of U.S. Provisional Application No. 60/537,898, filed Jan. 22, 2004, and U.S. Provisional Application No. 60/538,248, filed Jan. 23, 2004.

Disclosed herein are novel cosmetic compositions with one or more improved properties, intended for cleaning, conditioning, and/or styling keratin materials, such as the hair, and comprising, in a cosmetically acceptable medium, a washing base comprising at least one surfactant with detergent power, at least one nonaminated silicone, and at least one drawing polymer of high molecular weight. Also disclosed herein is the use of the compositions in a cosmetic application.

It is common practice to use detergent hair compositions (or shampoos) based, for example, essentially on standard anionic, nonionic and/or amphoteric surfactants, such as anionic surfactants, to clean and/or wash the hair. These compositions are applied to wet hair and the lather generated by massaging or rubbing with the hands removes, after rinsing with water, the various types of soiling initially present on the hair.

These base compositions may have good washing power, but the intrinsic cosmetic properties associated therewith nevertheless remain fairly poor. Poor cosmetic properties may be attributed to the relatively aggressive nature of such a cleaning treatment which can, in the long run, lead to more or less pronounced damage to the hair fiber, this damage being associated with the gradual removal of the lipids and/or proteins contained in or on the surface of this fiber.

Thus, in order to improve the cosmetic properties of the above detergent compositions, such as those that are intended to be applied to sensitized hair (i.e., hair that has been damaged or made brittle, for example due to the chemical action of atmospheric agents and/or hair treatments such as permanent-waving, dyeing, and/or bleaching), it is now common practice to introduce additional cosmetic agents known as conditioners into these compositions. These conditioners are intended mainly to repair or limit the harmful or undesirable effects induced by the various treatments or aggressions to which the hair fibers may be subjected more or less repeatedly. These conditioners may, of course, also improve the cosmetic behavior of natural hair.

The conditioners most commonly used in shampoos are cationic polymers, which give washed, dry, or wet hair an ease of disentangling, softness, and/or smoothness that may be better than those that may be obtained with corresponding cleaning compositions from which the conditioners are absent.

Moreover, it has for some time been sought to obtain conditioning shampoos that are capable of giving washed hair not only the cosmetic properties mentioned above, but also, to a greater or lesser extent, styling, volume, shaping, and/or hold properties. These washing shampoos with one or more improved general cosmetic properties may be referred to for simplicity as “styling shampoos”, and this term will be adopted in the description hereinbelow.

However, despite the progress made recently in the field of styling shampoos, these shampoos are not really completely satisfactory, and as such there is currently still a strong need to provide novel products that give better performance with respect to one or more of the cosmetic properties mentioned above. For example, it may be necessary to use a styling product after shampooing, to give the hair a shape and to fix the style.

The present disclosure is directed towards satisfying such a need.

Thus, after considerable research conducted in this matter, it has now been found that by combining at least one nonaminated silicone of a particular viscosity, as defined below, with at least one polymer that has a particular drawing power in detergent compositions, it may be possible to substantially and significantly improve the styling and hold properties, while at the same time maintaining good intrinsic washing power and cosmetic properties.

These compositions may make it possible to obtain very good hold and a certain amount of volume for the hair, i.e., a styling effect similar to that obtained with a fixing styling gel used after shampooing. It is moreover found that the keratin fibers are strengthened (hardened).

These discoveries form the basis of the present disclosure.

Thus, according to certain embodiments disclosed herein, novel compositions for washing keratin materials, such as the hair, are now proposed, comprising, in a cosmetically acceptable aqueous medium, at least one nonaminated silicone with a viscosity lower than 100,000 cst (mm²/s), at least one anionic, nonionic, or amphoteric detergent surfactant, and at least one polymer with a drawing power of greater than 5 cm.

One embodiment disclosed herein is also the cosmetic use of the above compositions for cleaning, conditioning, caring for, and styling keratin materials, such as the hair and the eyelashes.

Another embodiment disclosed herein comprises a cosmetic process for treating keratin materials, such as keratin fibers, for example the hair and the eyelashes, using the composition disclosed herein.

A further embodiment is the use of the composition disclosed herein as a shampoo.

However, other characteristics, aspects, and advantages will emerge even more clearly on reading the description that follows, as well as the concrete, but in no way limiting, examples intended to illustrate certain embodiments.

As used herein, the drawing power of a polymer corresponds to the length of the polymer yarn obtained at the breaking point of the yarn according to the procedure defined below.

The drawing power of the polymers that may be used according to the present disclosure may be the power measured for a composition containing (% by weight):

| | |
|---|---|
| Sodium lauryl ether sulphate oxyethylenated | 12.5% |
| with 2.2 mol of ethylene oxide | |
| Cocoamidopropylbetaine | 2.5% |
| Test polymer | 1% |
| Water qs | 100% |

The drawing power may be measured using a TA-TX2 texture analyser (Rheo/stable Micro Systems).

The measurement may be performed after compression of the product:
Displacement of the disc (35 mm aluminium cylinder) at a speed of 2.5 mm/s and detection of the compression strength;
penetration into the product at the same speed to a depth of 10 mm;
removal of the probe at a speed of 2.5 mm/s; and
measurement of the displacement of the probe and detection of the breaking point of the product yarn.

In the present disclosure, the polymers with a drawing power of greater than 5 cm will also be referred to as drawing polymers.

According to one embodiment, the polymers with a drawing power of greater than 5 cm may be either (a1) a dispersion of particles of at least one water-soluble cationic polymer with a weight-average molecular mass of greater than 10⁶ in a saline aqueous solution, obtained by heterogeneous free-radical polymerization of water-soluble monomers with precipitation of the polymer formed, or (a2) an aqueous solution of at least one water-soluble cationic polymer with a weight-average molecular mass of greater than 10⁶, obtained by heterogeneous free-radical polymerization of water-soluble monomers with precipitation of the polymer formed.

As used herein, the term “cationic polymer” includes polymers comprising cationic monomers and optionally comprising nonionic monomers.

The high molecular weight water-soluble cationic polymer can be a polyelectrolyte polymerized using at least one cationic monomer of formula (I), as defined below.

As indicated above, the synthesis of the high molecular weight water-soluble polymers used herein may take place by heterogeneous free-radical polymerization of water-soluble monomers comprising at least one ethylenic unsaturation. The polymerization may take place in an aqueous solution of a mineral electrolyte (salt) having an ionic strength that is sufficient to cause precipitation of the polymer formed as soon as it has reached a certain molecular mass. This polymerization technique thus allows, by virtue of the well-known phenomenon of salting out, the preparation of saline aqueous dispersions of water-soluble polymer particles. The polymers thus synthesized are distinguished by a high weight-average molecular mass, which may be greater than 10⁶.

The technique of heterogeneous free-radical polymerization in an aqueous medium with precipitation of the polymer formed is described, for example, in U.S. Pat. No. 4,929,655, in European Patent Application No. EP 0 943 628, and in PCT International Patent Application No. WO 02/34796.

To ensure the stability of the dispersions of polymer particles during the synthesis and during storage, one may perform the polymerization in the presence of a dispersant. This dispersant may be a polyelectrolyte, which, unlike the high molecular weight polymer used herein, is soluble in the aqueous polymerization medium of high ionic strength.

This dispersing polyelectrolyte may have a charge identical to that of the polymer synthesized. In other words, for the synthesis of cationic polyelectrolytes, a cationic dispersing polyelectrolyte may be used.

Dispersants that may be mentioned include the cationic polyelectrolytes obtained by polymerization of 50 to 100 mol % of at least one cationic monomer chosen from salts, such as hydrochlorides and sulphates of dimethylaminoethyl (meth)acrylate, of N-dimethylaminopropyl(meth)-acrylamide, of di(meth)allylamine, and of 50 to 100 mol % of acrylamide; (meth )acryloyloxy-ethyltrimethylammonium chloride; (meth )acrylamido-propyltrimethylammonium chloride; and dimethyldiallyl-ammonium chloride. A polyamine such as a polyalkyleneamine may also be used.

The dispersant may be used in an amount ranging from 1% to 10% by weight relative to the total weight of the monomers to be polymerized.

The saline aqueous solution that may serve as a synthesis and dispersion medium for the high molecular weight water-soluble polymer may be a solution of at least one mineral salt which may be chosen from divalent anionic salts. Examples of anionic salts that may be mentioned include ammonium sulphate, ammonium hydrogen sulphate, ammonium chloride, sodium sulphate, sodium hydrogen sulphate, magnesium sulphate, magnesium hydrogen sulphate, aluminium sulphate, and aluminium hydrogen sulphate. Ammonium sulphate and sodium sulphate, for example, may be mentioned.

The concentration of this at least one salt may be sufficient to induce the precipitation of the water-soluble polymer formed in the polymerization medium, and may be up to the saturation concentration of each salt. To obtain such a precipitation, the salt concentration may be at least equal to 10% by weight, such as greater than 15% by weight and less than 50% by weight relative to the total weight of the polymer solution or dispersion. The saline aqueous solution may also contain monovalent salts such as sodium chloride and ammonium chloride.

The heterogeneous free-radical polymerization in aqueous medium as described above may be accompanied by a large increase in the viscosity of the reaction medium, which may be reflected by difficulties in stirring, a lack of homogeneity of the reaction medium, and an increase in the particle size of the polymer particles formed. To prevent such an increase in viscosity, it has been proposed, for example in European Patent Application No. EP 0 943 628, to add to the polymerization medium at least one agent for preventing the increase in viscosity of the reaction medium during polymerization.

The high molecular weight water-soluble polymers used herein may be prepared in the presence of at least one such agent for preventing an increase in viscosity.

The at least one agent for preventing an increase in viscosity of the reaction medium may be chosen, for example, from:
(A) polycarboxylic acids and salts thereof,
(B) polyphenols,
(C) cyclic compounds containing at least one hydroxyl group and at least one carboxyl group, and salts thereof,
(D) gluconic acid and salts thereof,
(E) the reaction products obtained by reacting a methoxyhydroquinone and/or a cationic (meth)acrylic monomer with a free-radical-generating compound, under an oxidizing atmosphere,
(F) the reaction products obtained by reacting a cationic (meth)acrylic polymer with a free-radical-generating compound, under an oxidizing atmosphere, and
(G) the reaction products obtained by reacting a cationic (meth)acrylic polymer with an oxidizing agent.

The addition of at least one agent for preventing an increase in viscosity as described above may make it possible to perform the polymerization of the water-soluble monomers described above with a low-power stirrer while at the same time avoiding the formation of coarse particles. The at least one agent for preventing an increase in viscosity may be soluble in the aqueous reaction medium.

Examples of compounds (A) that may be mentioned include oxalic acid, adipic acid, tartaric acid, malic acid, phthalic acid, and the salts thereof.

Examples of compounds (B) that may be mentioned include resorcinol and pyrogallol.

Examples of compounds (C) that may be mentioned include m-hydroxybenzoic acid, p-hydroxybenzoic acid, salicylic acid, gallic acid, tannic acid, and the salts thereof.

Examples of compounds (D) that may be mentioned include sodium gluconate, potassium gluconate, ammonium gluconate, and various amine salts of gluconic acid.

Examples of compounds (E) that may be mentioned include those obtained by reacting a free-radical-generating compound, under a stream of oxygenated gas, in a solution containing methoxyhydroquinone and/or a cationic (meth)acrylic monomer. The free-radical-generating compound may be an initiator commonly used for free-radical polymerization. Examples that may be mentioned include water-soluble azo initiators such as 2,2′-azobis(2-amidinopropane) hydrochloride sold, for example, under the name V-50 by the company Wako Chemical Industries, and 2,2′-azobis[2-(2-imidazolin-2-yl)propane] hydrochloride sold, for example, under the name VA-044 by the company Wako Chemical Industries, and an initiator from the group of water-soluble redox agents, such as the ammonium persulphate/sodium hydrogen sulphite combination.

The at least one agent for preventing an increase in viscosity (F) may be obtained by reacting a free-radical initiator, under a oxygenated atmosphere, with a dispersant as disclosed herein. The polymerization initiator may be a water-soluble azo initiator or a water-soluble redox agent as described above.

The compounds (G) may be obtained in the form of oxidized polymers of low molecular mass by oxidation of at least one cationic dispersant as disclosed herein obtained by polymerization of a cationic (meth)acrylic monomer, using hydrogen peroxide or a halogen as oxidizing agent.

As cationic (meth)acrylic monomers used for the preparation of the at least one agent for preventing an increase in viscosity (E), (F), and (G), examples that may be mentioned include dimethylaminoethyl (meth)acrylate hydrochloride and sulphate, (meth)acryloyloxyethyl-trimethylammonium chloride, (meth )acryloyloxyethyl-dimethylbenzylammonium chloride, the hydrochloride and sulphate derived from N-dimethylaminopropyl(meth)-acrylamide, (meth)acrylamidopropyltrimethylammonium chloride, dimethylaminohydroxypropyl (meth)acrylate chloride and sulphate, (meth )acryloyloxyhydroxypropyl-trimethylammonium chloride, and (meth)acryloyloxyhydroxypropyldimethylbenzylammonium chloride.

The at least one agent for preventing an increase in viscosity (A) to (G) may be used alone or as a mixture, in an amount ranging, for example, from 10 ppm to 10,000 ppm, relative to the total weight of the reaction solution.

The water-soluble monomers polymerized by heterogeneous free-radical polymerization to obtain high molecular weight water-soluble cationic polymers are monomers comprising at least one ethylenic double bond, for example vinyl, acrylic, and allylic double bonds. They may be cationic or nonionic and may be used as a mixture.

Water-soluble nonionic monomers that may be mentioned include acrylamide, methacrylamide, N-vinylformamide, N-vinylacetonamide, hydroxypropyl acrylate, and hydroxypropyl methacrylate.

The water-soluble cationic monomers may be chosen from di(C₁₋₄ alkyl)diallylammonium salts and the compounds of formula (I) 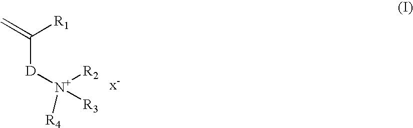
in which
R₁ is chosen from hydrogen and methyl groups,
R₂ and R₃, which may be identical or different, are each chosen from hydrogen and linear or branched C₁₋₄ alkyl groups,
R₄ is chosen from hydrogen, linear or branched C₁₋₄ alkyl groups, and aryl groups, and
D represents the following unit 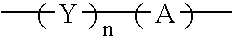
in which Y is chosen from amide (—CO—NH—), ester (—O—CO— or —CO—O—), urethane (—O—CO—NH—), and urea (—NH—CO—NH—) functions,
A is chosen from linear, branched, or cyclic C₁₋₁₀ alkylene groups, optionally substituted and optionally interrupted with a divalent aromatic or heteroaromatic ring, or optionally interrupted with a hetero atom chosen from O, N, S, and P, and which may comprise a functional group chosen from ketone, amide, ester, urethane, and urea functional groups,
n is chosen from 0 and 1, and
X⁻ is chosen from anionic counterions such as chloride and sulphate ions.

Examples of water-soluble cationic monomers that may be mentioned include dimethylaminoethyl (meth)acrylate hydrochloride and sulphate, (meth)acryloyloxyethyltrimethylammonium chloride, (meth)acryloyloxyethyldimethylbenzylammonium chloride, N-dimethylaminopropyl(meth)acrylamide hydrochloride and sulphate, (meth)acrylamidopropyltrimethylammonium chloride, (meth)acrylamidopropyldimethylbenzylammonium chloride, dimethylaminohydroxypropyl (meth)acrylate hydrochloride and sulphate, (meth)acryloyloxyhydroxypropyltrimethylammonium chloride, (meth)acryloyloxyhydroxypropyldimethylbenzylammonium chloride, and dimethyldiallylammonium chloride.

In one embodiment disclosed herein, the high molecular weight water-soluble polymer is obtained by heterogeneous free-radical polymerization of a monomer mixture comprising from 0 to 95.5 mol % of acrylamide and from 4.5 to 100 mol % of at least one cationic monomer of formula (I).

According to one embodiment, the water-soluble polymers are obtained by polymerization of a monomer mixture comprising acrylamide and at least one cationic monomer of formula (I), in which the number of moles of the at least one cationic monomer of formula (I) is greater than the number of moles of acrylamide.

Water-soluble cationic polymers that may be mentioned include, for example, those polymerized using monomer mixtures comprising, respectively:
1. 10 mol % of acryloyloxyethyldimethylbenzyl-ammonium chloride and 90 mol % of acrylamide;
2. 30 mol % of acryloyloxytrimethylammonium chloride, 50 mol % of acryloyloxyethyldimethylbenzyl-ammonium chloride and 20 mol % of acrylamide;
3. 10 mol % of acryloyloxyethyltrimethylammonium chloride and 90 mol % of acrylamide; or
4. 30 mol % of diallyldimethylammonium chloride and 70 mol % of acrylamide.

The water-soluble polymers used herein have a weight-average molecular mass of greater than 1,000,000, such as a mass ranging from 1,000,000 to 50,000,000 or ranging from 1,000,000 to 20,000,000. This weight-average molecular mass may be determined via the RSV (Reduced Specific Viscosity) method as defined in “Principles of Polymer Chemistry”, Cornell University Press, Ithaca, N.Y., 1953, chapter VII entitled “Determination of Molecular Weight”, pages 266-316.

The concentration of the high molecular weight water-soluble polymer dispersion or solution may be chosen such that the concentration of the water-soluble polymer ranges from 0.01% to 20% by weight, such as from 0.05% to 5%, by weight relative to the total weight of the final composition.

The concentration of the at least one drawing polymer disclosed herein may range from 0.01% to 10% by weight, such as from 0.05% to 5% by weight, relative to the total weight of the final composition.

The at least one detergent surfactant is chosen from anionic, amphoteric, nonionic, and zwitterionic surfactants.

Thus, according to the present disclosure, the at least one detergent surfactant may represent from 4% to 50% by weight, such as from 6% to 30% by weight or from 8% to 25% by weight, relative to the total weight of the final composition.

The at least one detergent surfactant that may be suitable for carrying out certain embodiments disclosed herein include the following:

(i) At Least One Anionic Surfactant:

As disclosed herein, the at least one anionic surfactant is optional.

Thus, as examples of the at least one anionic surfactant that may be used, mention may be made (as a non-limiting list) of salts (such as alkaline salts, for example sodium salts, ammonium salts, amine salts, amino alcohol salts, and magnesium salts) of the following compounds: alkyl sulphates, alkyl ether sulphates, alkylamido ether sulphates, alkylaryl polyether sulphates, monoglyceride sulphates, alkyl sulphonates, alkyl phosphates, alkylamide sulphonates, alkylaryl sulphonates, α-olefin sulphonates, paraffin sulphonates, alkyl sulphosuccinates, alkyl ether sulphosuccinates, alkylamide sulphosuccinates, alkyl sulphosuccinamates, alkyl sulphoacetates, alkyl ether phosphates, acyl sarcosinates, acyl isethionates, and N-acyltaurates, wherein the alkyl or acyl radical of all of these various compounds may have from 12 to 20 carbon atoms, and the aryl radical may be chosen from phenyl and benzyl groups. Among the at least one anionic surfactant that may be used, mention may also be made of fatty acid salts such as the salts of oleic, ricinoleic, palmitic, and stearic acids; coconut oil acid; hydrogenated coconut oil acid; and acyl lactylates in which the acyl radical contains 8 to 20 carbon atoms. Use may also be made of at least one weakly anionic surfactant, such as alkyl-D-galactosiduronic acids and their salts, and polyoxyalkylenated carboxylic ether acids and their salts, such as those containing from 2 to 50 ethylene oxide groups. Anionic surfactants of the polyoxyalkylenated carboxylic ether acid or salt type may, for example, correspond to formula (1) below:
R₁-(OC₂H₄)ₙ—OCH₂COOA (1)
in which:
R₁ is chosen from alkyl, alkylamido, and alkaryl groups, and n is chosen from integers and decimal numbers (average value) that may range from 2 to 24, such as from 3 to 10, wherein the alkyl radical has between 6 and 20 carbon atoms approximately, and the aryl radical may be a phenyl,
A is chosen from hydrogen, ammonium, Na, K, Li, Mg, monoethanolamine, and triethanolamine residues. Mixtures of compounds of formula (1) can also be used, for example mixtures in which the groups R₁ are different.

Compounds of formula (1) are sold, for example, by the company Chem Y under the name Akypo® (NP40, NP70, OP40, OP80, RLM25, RLM38, RLMQ 38 NV, RLM 45, RLM 45 NV, RLM 100, RLM 100 NV, RO 20, RO 90, RCS 60, RS 60, RS 100, and RO 50) and by the company Sandoz under the name Sandopan® (DTC Acid and DTC).

(ii) At Least One Nonionic Surfactant:

Nonionic surfactants are likewise compounds that are well known per se (see in this respect “Handbook of Surfactants” by M. R. Porter, published by Blackie & Son (Glasgow and London), 1991, pp. 116-178) and, as used herein, the at least one nonionic surfactant is optional. Thus, it may be chosen from (as a non-limiting list) polyethoxylated, polypropoxylated, and polyglycerolated fatty alcohols; polyethoxylated, polypropoxylated, and polyglycerolated fatty α-diols; polyethoxylated, polypropoxylated, and polyglycerolated fatty alkylphenols; and polyethoxylated, polypropoxylated, and polyglycerolated fatty acids, all having a fatty chain containing, for example, 8 to 18 carbon atoms, it being possible for the number of ethylene oxide or propylene oxide groups to range, for example, from 2 to 50 and for the number of glycerol groups to range, for example, from 2 to 30. Mention may also be made of copolymers of ethylene oxide and of propylene oxide; condensates of ethylene oxide and of propylene oxide with fatty alcohols; polyethoxylated fatty amides, for example polyoethoxylated fatty amides having from 2 to 30 mol of ethylene oxide; polyglycerolated fatty amides having on average 1 to 5, such as 1.5 to 4, glycerol groups; oxyethylenated fatty acid esters of sorbitan having from 2 to 30 mol of ethylene oxide; fatty acid esters of sucrose; fatty acid esters of polyethylene glycol alkylpolyglycosides; N-alkylglucamine derivatives; amine oxides such as (C₁₀-C₁₄)alkylamine oxides; and N-acylaminopropylmorpholine oxides. The alkylpolyglycosides may be mentioned as nonionic surfactants that are suitable in the context of the present disclosure.

(iii) At Least One Amphoteric or Zwitterionic Surfactant:

The at least one amphoteric or zwitterionic surfactant, which is optional according to embodiments disclosed herein, can be, for example (as a non-limiting list), aliphatic secondary and tertiary amine derivatives in which the aliphatic radical is a linear or branched chain containing 8 to 18 carbon atoms and containing at least one water-soluble anionic group (for example carboxylate, sulphonate, sulphate, phosphate and phosphonate groups); mention may also be made of (C₈-C₂₀)alkylbetaines, sulphobetaines, (C₈-C₂₀)alkylamido (C₁-C₆)alkylbetaines, and (C₈-C₂₀)alkylamido(C₁-C₆)alkylsulphobetaines.

Among the amine derivatives, mention may be made of the products sold under the name Miranol®, as described in U.S. Pat. Nos. 2,528,378 and 2,781,354 and classified in the CTFA dictionary, 3rd edition, 1982, under the names Amphocarboxyglycinates and Amphocarboxypropionates, with the respective structures (2) and (3):
R₂-CONHCH₂CH₂—N⁺(R₃)(R₄)(CH₂COO⁻) (2)
in which: R₂ is chosen from alkyl radicals of an acid R₂-COOH present in hydrolysed coconut oil, heptyl radicals, nonyl radicals, and undecyl radicals, R₃ denotes a β-hydroxyethyl group, and R₄ denotes a carboxymethyl group;
and
   R_{2′}-CONHCH₂CH₂—N(B)(D) (3)
in which:
   B represents —CH₂CH₂OX′, D represents —(CH₂)₂-Y′, wherein z is chosen from 1 and 2,
   X′ chosen from —CH₂CH₂—COOH and hydrogen,
   Y′ is chosen from —COOH and —CH₂—CHOH—SO₃H,
   R₂′ is chosen from alkyl radicals, such as alkyl radicals of an acid R₂-COOH present in coconut oil or in hydrolysed linseed oil; C₇, C₉, C₁₁, C₁₃ alkyl radicals, C₁₇ alkyl radicals and its iso form; and unsaturated C₁₇ radicals.

For example, mention may be made of the cocoamphocarboxyglycinate sold under the trade name Miranol® C2M concentrate by the company Miranol.

Mixtures of surfactants, for example mixtures of anionic surfactants, mixtures of anionic surfactants and of amphoteric, cationic or nonionic surfactants, and mixtures of cationic surfactants with nonionic or amphoteric surfactants, may be used in the compositions as disclosed herein. A mixture that may be mentioned is a mixture comprising at least one anionic surfactant and at least one amphoteric surfactant.

The amount of the at least one anionic surfactant ranges from 4% to 50% by weight relative to the total weight of the cosmetic composition. It may, for example, range from 5% to 35% by weight or from 8% to 25% by weight, relative to the total weight of the cosmetic composition.

The amount of the at least one amphoteric and/or nonionic surfactant, when it is present, may range from 0.5% to 20% by weight, such as from 1% to 15% by weight, relative to the total weight of the composition.

In general, as used herein, the term “nonaminated silicone” denotes any silicone not containing at least one primary, secondary, or tertiary amine or one quaternary ammonium group.

Nonaminated silicones having a viscosity of less than 100,000 mm²/s which can be used in accordance with certain embodiments may include polyorganosiloxanes that are insoluble in the composition and may be in the form of oils, waxes, and resins. The silicones may have a viscosity ranging from 1,000 to 100,000 cst, such as from 10,000 to 80,000 cst.

The organopolysiloxanes are defined in greater detail in Walter NOLL “Chemistry and Technology of Silicones” (1968) Academie Press.

Non-volatile silicones may be used according to certain embodiments, including for example at least one of polydialkylsiloxanes, polydiarylsiloxanes, polyalkylarylsiloxanes, silicone gums, silicone resins, and polyorganosiloxanes modified with organofunctional groups.

These silicones, may, for example, be chosen from polyalkylsiloxanes, among which mention may be made of polydimethylsiloxanes containing trimethylsilyl end groups and having a viscosity ranging from 100 to 100,000 mm²/s (cSt) at 25° C., such as from 1,000 to 100,000 mm²/s (cSt) or from 10,000 to 80,000 mm2/s (cSt). The viscosity of the silicones may be measured, for example, at 25° C. according to ASTM standard 445 Appendix C.

Among these polyalkylsiloxanes, mention may be made, in a non-limiting manner, of the following commercial products:
the oils of Mirasil® series sold by Rhodia, such as, for example, the oil Mirasil® DM 500;
the oils of the 200 series from the company Dow Corning, such as, for example, DC200 with a viscosity of 60,000 cSt;
the Viscasil® oils from General Electric and certain oils of the SF series (SF 96, SF 18) from General Electric.

Mention may also be made of polydimethylsiloxanes containing dimethylsilanol end groups, known under the name dimethiconol (CTFA), such as the oils of the 48 series from the company Rhône Poulenc.

In this category of polydialkylsiloxanes, mention may also be made of the products sold under the names Abil® Wax 9800 and 9801 by the company Goldschmidt, which are poly(C₁-C₂₀)dialkylsiloxanes.

The polyalkylarylsiloxanes may be chosen from polydimethyl/methylphenylsiloxanes, linear and/or branched polydimethyldiphenylsiloxanes with a viscosity ranging from 10 mm²/s to 5,000 mm²/s at 25° C., such as a viscosity ranging from 100 mm²/s to 5,000 mm²/s.

Among these polyalkylarylsiloxanes, examples that may be mentioned include the products sold under the following names:
the Silbione® oils of the series 70 641 from Rhône Poulenc;
the oils of the Rhodorsil® 70 633 and 763 series from Rhône Poulenc;
the oil Dow Corning 556 Cosmetic Grade Fluid from Dow Corning;
the silicones of the PK series from Bayer, for instance the product PK20;
the silicones of the PN and PH series from Bayer, for instance the products PN1000 and PH1000; and
certain oils of the SF series from General Electric, such as SF 1023, SF 1154, SF 1250 and SF 1265.

The organomodified silicones that can be used in accordance with certain embodiments are silicones as defined above and containing in their structure one or more organofunctional groups attached via a hydrocarbon-based radical.

Among the organomodified silicones, mention may be made of polyorganosiloxanes comprising:
thiol groups such as the products sold under the names GP 72 A and GP 71 from Genesee;
alkoxylated groups such as the product sold under the name Silicone Copolymer F-755 by SWS Silicones and Abil® Wax 2428, 2434, and 2440 by the company Goldschmidt;
hydroxylated groups such as the polyorgano-siloxanes containing a hydroxyalkyl function, described in French Patent Application No. FR A 85/16334;
acyloxyalkyl groups such as, for example, the polyorganosiloxanes described in U.S. Pat. No.4,957,732;
carboxylic anionic groups, such as, for example, in the products described in European Patent No. EP 186 507 from the company Chisso Corporation; alkylcarboxylic anionic groups, such as those present in the product X-22-3701 E from the company Shin-Etsu; 2-hydroxyalkyl sulfonate; 2-hydroxyalkyl thiosulfate such as the products sold by the company Goldschmidt under the names Abil® S201 and Abil® S255.

According to certain embodiments, it is also possible to use silicones comprising a polysiloxane portion and a portion comprising a non-silicone organic chain, one of the two portions constituting the main chain of the polymer and the other being grafted onto the main chain. These polymers are described for example in Patent Application Nos. EP A 412 704, EP A 412 707, EP A 640 105, WO 95/00578, EP A 582 152, and WO 93/23009 and U.S. Pat. Nos. 4,693,935, 4,728,571, and 4,972,037. These polymers may be anionic or non-ionic.

Such polymers are for example the copolymers which can be obtained by free-radical polymerization from the mixture of monomers comprising:
a) 50 to 90% by weight of tert-butyl acrylate;
b) 0 to 40% by weight of acrylic acid;
c) 5 to 40% by weight of silicone macromer of formula: 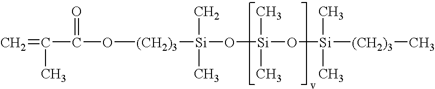
   with v being a number ranging from 5 to 700; the percentages by weight being calculated relative to the total weight of the monomers.

Other examples of graft silicone polymers include polydimethylsiloxanes (PDMS) onto which mixed polymer units of the poly(meth)acrylic acid type and of the polyalkyl (meth)acrylate type are grafted via a connecting member of the thiopropylene type and polydimethylsiloxanes (PDMS) onto which polymer units of the polyisobutyl (meth)acrylate type are grafted via a connecting member of the thiopropylene type.

According to certain embodiments, the silicones may also be used in the form of emulsions, nanoemulsions, and microemulsions.

The polyorganosiloxanes that may be mentioned include:
the nonvolatile silicones chosen from the family of polydialkylsiloxanes containing terminal trimethylsilyl groups such as the oils having a viscosity ranging from 1,000 to 100,000 mm²/s (cSt) at 25° C., such as the oils of the DC200 series from Dow Corning, for example the oil with a viscosity of 60,000 mm²/s (cSt), of the Mirasil® DM series, polydialkylsiloxanes with terminal dimethylsilanol groups such as dimethiconol or polyalkylarylsiloxanes such as the oil Mirasil® DPDM sold by the company Rhodia Chimie.

According to certain embodiments, the at least one nonaminated silicone may represent in an amount ranging from 0.001% to 20% by weight, such as from 0.01% to 10% by weight or from 0.1% to 3% by weight, relative to the total weight of the final composition.

According to one embodiment, the compositions may also comprise at least one cationic polymer other than the at least one drawing polymer disclosed herein.

The at least one cationic polymer that may be used may be chosen from those already known per se as improving the cosmetic properties of the hair, i.e., those described in European Patent Application No. EP A 0 337 354 and in French Patent Application Nos. FR A 2 270 846, 2 383 660, 2 598 611, 2 470 596, and 2 519 863, and having a suitable cationic charge density.

As used herein, the term “cationic polymer” denotes any polymer comprising cationic groups and/or groups that may be ionized into cationic groups.

Mention may be made of the cationic polymers that are chosen from those containing units comprising primary, secondary, tertiary, and/or quaternary amine groups that either may form part of the main polymer chain or may be borne by a side substituent directly attached thereto.

The at least one cationic polymer used may have a number-average or weight-average molar mass ranging from 500 to5×10^{6,} such as from 10³ to 3×10^{6.}

Among the cationic polymers that may be mentioned are polymers of the polyamine, polyamino amide, and polyquaternary ammonium type. These are known products.

The polymers of the polyamine, polyamido amide, and polyquaternary ammonium type that may be used in accordance with certain embodiments include those described, for example, in French Patent Nos. 2 505 348 and 2 542 997. Among these polymers, mention may be made of:
(1) homopolymers or copolymers derived from acrylic or methacrylic esters or amides and comprising at least one of the units of the following formulae: 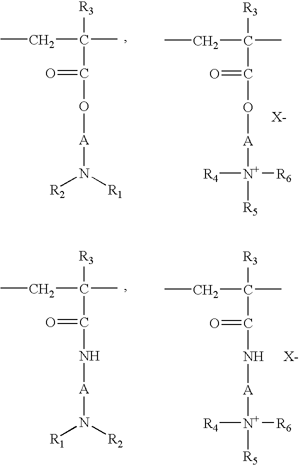
in which:
   R₃, which may be identical or different, is chosen from hydrogen and CH₃ radicals;
   A, which may be identical or different, is chosen from linear or branched alkyl groups having 1 to 6 carbon atoms, such as 2 or 3 carbon atoms and hydroxyalkyl groups having 1 to 4 carbon atoms;
   R₄, R₅, and R₆, which may be identical or different, are chosen from alkyl groups containing from 1 to 18 carbon atoms and benzyl radicals. For example R₄, R₅, and R₆, which may be identical or different, are chosen from alkyl groups containing from 1 to 6 carbon atoms;
   R₁ and R₂, which may be identical or different, are chosen from hydrogen and alkyl groups containing from 1 to 6 carbon atoms, such as methyl and ethyl groups;
   X⁻ denotes an anion derived from a mineral or organic acid, such as an anion chosen from methosulphate anions, and halide anions, such as chloride and bromide.

The copolymers of this group (1) may also comprise one or more units derived from comonomers that may be chosen from the family of acrylamides, methacrylamides, diacetone acrylamides, acrylamides, and methacrylamides substituted on the nitrogen with at least one of lower (C₁-C₄) alkyls, acrylic or methacrylic acids or esters thereof, vinyllactams such as vinylpyrrolidone and vinylcaprolactam, and vinyl esters.

Thus, among these copolymers of group (1), mention may be made of:
copolymers of acrylamide and of dimethylaminoethyl methacrylate quaternized with at least one of dimethyl sulphates and dimethyl halides, such as the product sold under the name Hercofloc® by the company Hercules,
copolymers of acrylamide and of methacryloyloxyethyltrimethylammonium chloride described, for example, in European Patent Application No. EP A 080 976 and sold under the name Bina Quat P 100 by the company Ciba Geigy,
copolymers of acrylamide and of methacryloyloxyethyltrimethylammonium methosulphate sold under the name Reten® by the company Hercules,
quaternized or non-quaternized vinylpyrrolidone/ dialkylaminoalkyl acrylate or methacrylate copolymers. These polymers are described, for example, in detail in French Patent Nos. 2 077 143 and 2 393 573,
dimethylaminoethyl methacrylate/vinylcaprolactam/ vinylpyrrolidone terpolymers,
vinylpyrrolidone/methacrylamidopropyldimethylamine copolymers, and
quaternized vinylpyrrolidone/dimethylaminopropylmethacrylamide copolymers.

(2) cationic polysaccharides, such as cationic celluloses and cationic galactomannan gums. Among the cationic polysaccharides that may be mentioned are cellulose ether derivatives comprising quaternary ammonium groups, cationic cellulose copolymers, cellulose derivatives grafted with a water-soluble quaternary ammonium monomer, and cationic galactomannan gums.

The cellulose ether derivatives comprising quaternary ammonium groups are described, for example, in French Patent No.1 492 597. These polymers are also defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are described for example in U.S. Pat, No. 4,131,576, such as hydroxyalkylcelluloses, for instance hydroxymethyl-, hydroxyethyl-, or hydroxypropylcelluloses grafted, for example, with a methacryloylethyltrimethylammonium, methacrylamidopropyltrimethylammonium or dimethyldiallylammonium salt.

The cationic galactomannan gums are described for example in U.S. Pat. Nos. 3,589,578 and 4,031,307, for example guar gums containing trialkylammonium cationic groups. Use is made, for example, of guar gums modified with a salt (e.g., chloride) of 2,3-epoxypropyltrimethylammonium.

(3) polymers comprising piperazinyl units and divalent alkylene or hydroxyalkylene radicals containing straight or branched chains, optionally interrupted by at least one of oxygen, sulphur, nitrogen, and aromatic or heterocyclic rings, and also the oxidation and/or quaternization products of these polymers. Such polymers are described, for example, in French Patent Nos. 2 162 025 and 2 280 361.

(4) water-soluble polyamino amides prepared, for instance, by polycondensation of an acidic compound with a polyamine. These polyamino amides can be crosslinked with at least one group chosen from epihalohydrins, diepoxides, dianhydrides, unsaturated dianhydrides, bis-unsaturated derivatives, bis-halohydrins, bis-azetidiniums, bis-haloacyidiamines, bis-alkyl halides, and oligomers resulting from the reaction of a difunctional compound that is reactive with respect to at least one group chosen from bis-halohydrins, bis-azetidiniums, bis-haloacyldiamines, bis-alkyl halides, epihalohydrins, diepoxides, and bis-unsaturated derivatives; the crosslinking agent being used in proportions ranging from 0.025 to 0.35 mol per amine group of the polyamino amide. These polyamino amides can be alkylated or, if they contain one or more tertiary amine functions, they can be quaternized. Such polymers are described, for example, in French Patent Nos. 2 252 840 and 2 368 508.

(5) polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical contains from 1 to 4 carbon atoms, such as methyl, ethyl, and propyl radicals. Such polymers are described for example in French Patent No.1 583 363.

Among these derivatives, mention may be made of the adipic acid/dimethylaminohydroxypropyl/diethylenetriamine polymers sold under the name Cartaretine F, F4 or F8 by the company Sandoz.

(6) polymers obtained by reaction of a polyalkylene polyamine containing two primary amine groups and at least one secondary amine group with a dicarboxylic acid chosen from diglycolic acid and saturated aliphatic dicarboxylic acids containing from 3 to 8 carbon atoms. The molar ratio between the polyalkylene polyamine and the dicarboxylic acid may range from 0.8:1 to 1.4:1. The polyamino amide resulting therefrom may be reacted with epichlorohydrin in a molar ratio of epichlorohydrin relative to the secondary amine group of the polyamino amide ranging from 0.5:1 to 1.8:1. Such polymers are described for example in U.S. Pat. Nos. 3,227,615 and 2,961,347.

Polymers of this type are sold for example under the name Hercosett® 57 by the company Hercules Inc., in the case of the adipic acid/epoxypropyl/diethylenetriamine copolymer.

(7) cyclopolymers of alkyldiallylamine or of dialkyldiallylammonium, such as the homopolymers or copolymers containing, as main constituent of the chain, units corresponding to formula (I′) or (I″): 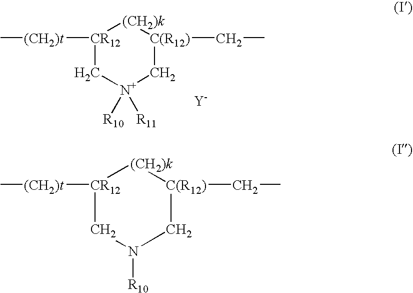
in which formulae k and t are chosen from 0 and 1, the sum k+t being equal to 1; R₁₂ is chosen from hydrogen and methyl radicals; R₁₀ and R₁₁, independently of each other, are chosen from alkyl groups containing from 1 to 6 carbon atoms, hydroxyalkyl groups in which the alkyl group may contain 1 to 5 carbon atoms, andlower (C₁-C₄) amidoalkyl groups, or R₁₀ and R₁₁ can be chosen from, together with the nitrogen atom to which they are attached, heterocyclic groups such as piperidyl and morpholinyl groups; Y⁻ is an anion such as bromide, chloride, acetate, borate, citrate, tartrate, bisulphate, bisulphite, sulphate, and phosphate. These polymers are described for example in French Patent No. 2 080 759 and in its Certificate of Addition 2 190 406.

In one embodiment, R₁₀ and R₁₁, independently of each other, may denote an alkyl group containing from 1 to 4 carbon atoms.

Among the polymers defined above, mention may be made of the dimethyldiallylammonium chloride homopolymer sold under the name Merquat® 100 by the company Nalco (and its homologues of low weight-average molar mass) and copolymers of diallyldimethylammonium chloride and of acrylamide.

(8) diquaternary ammonium polymers containing repeating units corresponding to the formula: 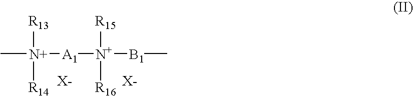
in which formula (II):
R₁₃, R₁₄, R₁₅, and R₁₆, which may be identical or different, are chosen from aliphatic, alicyclic or arylaliphatic radicals containing from 1 to 20 carbon atoms and lower hydroxyalkylaliphatic radicals, or alternatively R₁₃, R₁₄, R₁₅, and R₁₆, together or separately, constitute, together with the nitrogen atoms to which they are attached, heterocycles optionally containing a second hetero atom other than nitrogen, or alternatively R₁₃, R₁₄, R₁₅, and R₁₆ represent a linear or branched C₁-C₆ alkyl radical substituted with a group chosen from nitrile groups, ester groups, acyl groups, amide groups, and groups —CO—O-R₁₇-D and —CO—NH-R₁₇-D where R₁₇ is an alkylene and D is a quaternary ammonium group;
A₁ and B₁ represent polymethylene groups containing from 2 to 20 carbon atoms, which groups may be linear or branched, saturated or unsaturated, and which may contain, linked to or intercalated in the main chain, one or more aromatic rings or one or more groups chosen from oxygen, sulphur, sulphoxide, sulphone, disulphide, amino, alkylamino, hydroxyl, quaternary ammonium, ureido, amide, and ester groups, and
X⁻ denotes an anion derived from a mineral or organic acid;
A₁, R₁₃, and R₁₅ can form, together with the two nitrogen atoms to which they are attached, a piperazine ring; in addition, if A₁ is chosen from linear or branched, saturated or unsaturated alkylene and hydroxyalkylene radicals, B₁ can also denote a group (CH₂)ₙ—CO-E-OC—(CH₂)ₙ—
in which E is chosen from:
   a) glycol residues of formula: —O-Z-O—, where Z is chosen from linear or branched hydrocarbon-based radicals and groups corresponding to one of the following formulae:
      —(CH₂—CH₂—O)ₓ—CH₂—CH₂—
      —[CH₂—CH(CH₃)—O]_{y}—CH₂—CH(CH₃)—
   where x and y are chosen from integers ranging from 1 to 4, representing a defined and unique degree of polymerization and any number ranging from 1 to 4 representing an average degree of polymerization;
   b) bis-secondary diamine residues such as piperazine derivatives;
   c) bis-primary diamine residues of formula: —NH-Y-NH—, where Y is chosen from linear or branched hydrocarbon-based radicals and divalent radicals
      —CH₂—CH₂—S—S—CH₂—CH₂—; and
   d) ureylene groups of formula: —NH—CO—NH—.

In certain embodiments, X⁻ is an anion such as chloride and bromide.

These polymers may have a number-average molar mass ranging from 1,000 to 100,000.

Polymers of this type are described for example in French Patent Nos. 2 320 330, 2 270 846, 2 316 271, 2 336 434, and 2 413 907 and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945; and 4,027,020.

It may also be possible to use polymers that comprise repeating units corresponding to the formula: 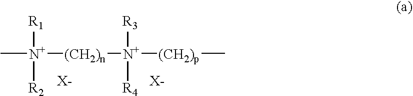
in which R₁, R₂, R₃, and R₄, which may be identical or different, are chosen from alkyl and hydroxyalkyl radicals containing from 1 to 4 carbon atoms approximately, n and p are chosen from integers ranging from 2 to 20 approximately, and X⁻ is an anion derived from a mineral or organic acid.

One compound of formula (a) which may be mentioned is the one for which R₁, R₂, R₃, and R₄ represent a methyl radical, n is equal to 3, p is equal to 6, and X is equal to Cl, which compound is known as Hexadimethrine chloride according to the INCI (CTFA) nomenclature.

(9) polyquaternary ammonium polymers comprising units of formula (III):

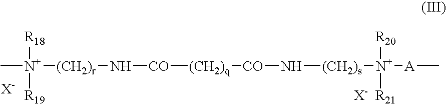

in which formula:
R₁₈, R₁₉, R₂₀, and R₂₁, which may be identical or different, are chosen from hydrogen, methyl radicals, ethyl radicals, propyl radicals, β-hydroxyethyl radicals, β-hydroxypropyl radicals, and —CH₂CH₂(OCH₂CH₂)ₚOH radicals,
where p is chosen from 0 or integers ranging from 1 to 6, with the proviso that R₁₈, R₁₉, R₂₀, and R₂₁ do not simultaneously represent hydrogen,
r and s, which may be identical or different, are chosen from integers ranging from 1 to 6,
q is chosen from 0 and integers ranging from 1 to 34,
X⁻ denotes an anion such as a halide,
A is chosen from dihalide radicals and may represent —CH₂—CH₂—O—CH₂—CH₂—.

Such compounds are described for example in European Patent Application No. EP A 122 324.

Among these products, mention may be made, for example, of the products Mirapol® A 15, Mirapol® AD1, Mirapol® AZ1, and Mirapol® 175 sold by the company Miranol.

(10) quaternary polymers of vinylpyrrolidone and of vinylimidazole, for instance the products sold under the names Luviquat® FC 905, FC 550 and FC 370 by the company BASF; and

(11) crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, such as methylenebisacrylamide.

Other cationic polymers that may be used in accordance with certain embodiments disclosed herein are cationic proteins or cationic protein hydrolysates, polyalkyleneimines, such as polyethyleneimines, polymers containing vinylpyridine or vinylpyridinium units, condensates of polyamines and of epichlorohydrin, polyquaternary ureylenes and chitin derivatives, such as chitosans, and salts thereof;

The salts that may be used include, for example, chitosan acetate, lactate, glutamate, gluconate, and pyrrolidonecarboxylate.

Among these compounds, mention may be made of chitosan with a degree of deacetylation of 90% by weight, and the chitosan pyrrolidonecarboxylate sold under the name Kytamer® PC by the company Amerchol.

Among all the cationic polymers that may be used in the context of embodiments disclosed herein, mention may be made of the use of cationic cyclopolymers, such as the dimethyldiallylammonium chloride homopolymers and copolymers sold under the names Merquat 100®, Merquat® 550, and Merquat® S by the company Nalco, quaternary polymers of vinylpyrrolidone and of vinylimidazole, crosslinked homopolymers and copolymers of methacryloyloxy(C₁-C₄)alkyltri(C₁-C₄)alkylammonium salts, and the chitosan pyrrolidonecarboxylate, and mixtures thereof.

The amphoteric polymers that may be used in accordance with certain embodiments may be chosen from polymers comprising units K and M randomly distributed in the polymer chain, in which K denotes a unit derived from a monomer comprising at least one basic nitrogen atom and M denotes a unit derived from an acidic monomer comprising one or more groups chosen from carboxylic groups and sulphonic groups, or alternatively K and M may denote groups derived from zwitterionic carboxybetaine or sulphobetaine monomers;

K and M may also denote a cationic polymer chain comprising primary, secondary, tertiary, or quaternary amine groups, in which at least one of the amine groups bears a carboxylic or sulphonic group linked via a hydrocarbon-based radical, or alternatively K and M may form part of a chain of a polymer containing an α,β-dicarboxylic ethylene unit in which one of the carboxylic groups has been made to react with a polyamine comprising one or more primary or secondary amine groups.

The amphoteric polymers corresponding to the above definition that may be mentioned are chosen from the following polymers:
(1) polymers resulting from the copolymerization of a monomer derived from a vinyl compound bearing a carboxylic group such as acrylic acid, methacrylic acid, maleic acid, α-chloroacrylic acid, and a basic monomer derived from a substituted vinyl compound containing at least one basic atom, such as dialkylaminoalkyl methacrylate and acrylate, dialkylaminoalkylmethacrylamide and -acrylamide. Such compounds are described, for example, in U.S. Pat. No. 3,836,537. Mention may also be made of the sodium acrylate/acrylamidopropyltrimethylammonium chloride copolymer sold under the name Polyquart® KE 3033 by the company Cognis.
The vinyl compound may also be a dialkyldiallylammonium salt such as dimethyldiallylammonium salt (for example chloride). The copolymers of acrylic acid and of the latter monomer are sold, for example, under the names Merquat® 280 and Merquat® 295 by the company Nalco.
(2) Polymers containing units derived from:
   a) at least one monomer chosen from acrylamides and methacrylamides substituted on the nitrogen with an alkyl radical,
   b) at least one acidic comonomer containing one or more reactive carboxylic groups, and
   c) at least one basic comonomer such as esters containing primary, secondary, tertiary, or quaternary amine substituents of acrylic and methacrylic acids and the product of quaternization of dimethylaminoethyl methacrylate with dimethyl or diethyl sulphate.
The N-substituted acrylamides or methacrylamides that may be mentioned are groups in which the alkyl radicals contain from 2 to 12 carbon atoms, such as N-ethylacrylamide, N-tert-butylacrylamide, N-tert-octylacrylamide, N-octylacrylamide, N-decylacrylamide, N-dodecylacrylamide, and the corresponding methacrylamides.
The acidic comonomers may be chosen from acrylic acid, methacrylic acid, crotonic acid, itaconic acid, maleic acid, fumaric acid, alkyl monoesters, having 1 to 4 carbon atoms, of maleic and fumaric acids and anhydrides.
As basic comonomers, mention may be made of aminoethyl, butylaminoethyl, N,N′-dimethylaminoethyl, and N-tert-butylaminoethyl methacrylates.
The copolymers whose CTFA (4th edition, 1991) name is Octylacrylamide/acrylates/butylaminoethyl methacrylate copolymer may be used.
(3) Polyamino amides that are crosslinked and alkylated partially or totally derived from polyamino amides of general formula: 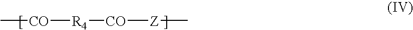
in which R₄ is chosen from divalent radicals derived from a saturated dicarboxylic acid, mono- and dicarboxylic aliphatic acids containing an ethylenic double bond, esters of a lower alkanol containing 1 to 6 carbon atoms of these acids, and radicals derived from the addition of any one of the said acids to a bis(primary) or bis(secondary) amine; and Z is chosen from bis(primary), mono-, and bis(secondary) polyalkylene-polyamine radicals and may, for example represent:
   a) in an amount ranging from 60 to 100 mol %, the radical 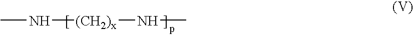
where x is 2 and p is chosen from 2 and 3, or alternatively x is 3 and p is 2,
this radical being derived from a group chosen from diethylenetriamine, triethylenetetraamine, and dipropylenetriamine;
   b) in an amount ranging from 0 to 40 mol %, the radical (V) above in which x is 2 and p is 1 and which is derived from ethylenediamine, or the radical derived from piperazine: 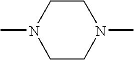
   c) in an amount ranging from 0 to 20 mol %, the —NH—(CH₂)₆—NH— radical derived from hexamethylenediamine, these polyamino amines being crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of at least one group chosen from acrylic acid, chloroacetic acid, alkane sultone, and salts thereof.
The saturated carboxylic acids may be chosen from acids having 6 to 10 carbon atoms, such as adipic acid, 2,2,4-trimethyladipic acid, 2,4,4-trimethyladipic acid, terephthalic acid, and acids containing an ethylenic double bond such as, for example, acrylic acid, methacrylic acid, and itaconic acid.
The alkane sultones used in the alkylation may, for example, be chosen from propane sultone, and butane sultone, and the salts of the alkylating agents may be chosen from sodium and potassium salts.
(4) Polymers containing zwitterionic units of formula: 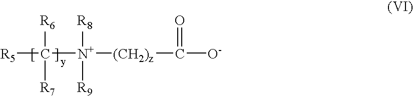
in which R₅ denotes a polymerizable unsaturated group such as acrylate, methacrylate, acrylamide, and methacrylamide groups; y and z are chosen from integers ranging from 1 to 3; R₆ and R₇ are chosen from hydrogen, methyl, ethyl, and propyl groups, R₈ and R₉ are chosen from hydrogen and alkyl radicals such that the sum of the carbon atoms in R₈ and R₉ does not exceed 10.

The polymers comprising such units may also comprise units derived from non-zwitterionic monomers such as dimethyl and diethylaminoethyl acrylate, dimethyl and diethylaminoethyl methacrylate, alkyl acrylates, alkyl methacrylates, acrylamides, methacrylamides, and vinyl acetate.

By way of example, mention may be made of the copolymer of butyl methacrylate/dimethylcarboxymethylammonioethyl methacrylate.
(5) Polymers derived from chitosan comprising monomer units corresponding to formulae (VII), (VIII), and (IX) below: 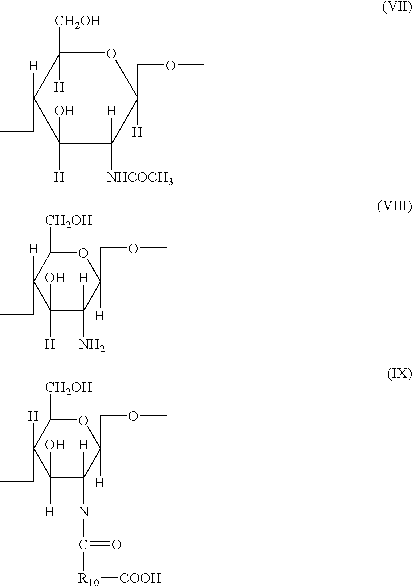
the unit (VII) being present in an amount ranging from 0 to 30%, the unit (VIII) being present in an amount ranging from 5 to 50%, and the unit (IX) being present in an amount ranging from 30 to 90%, it being understood that, in this unit (IX), R₁₀ represents a radical of formula: 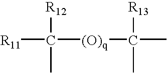
in which
   if q is 0, R₁₁, R₁₂, and R₁₃, which may be identical or different, are each chosen from hydrogen, methyl residues, hydroxyl residues, acetoxy residues, amino residues, monoalkylamine residues, dialkylamine residues that are optionally interrupted by at least one nitrogen atom and optionally substituted with at least one residue chosen from amine, hydroxyl, carboxyl, alkylthio, and sulphonic groups, and alkylthio residues in which the alkyl group bears an amino residue, at least one of the radicals R₁₁, R₁₂, and R₁₃ being, in this case, hydrogen;
   or, if q is 1, R₁₁, R₁₂, and R₁₃ each represent a hydrogen atom, and also the acid or base salts formed by these compounds.
(6) Polymers derived from the N-carboxyalkylation of chitosan, such as N-carboxymethylchitosan and N-carboxybutylchitosan.
(7) Polymers corresponding to the general formula (X) such as those described, for example, in French Patent No. 1 400 366: 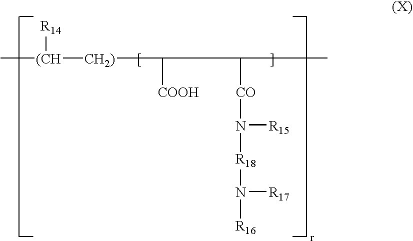
in which
   r is an integer greater than 1;
   R₁₄ is chosen from hydrogen, CH₃O, CH₃CH₂O, and phenyl radicals;
   R₁₅ is chosen from hydrogen and lower alkyl radicals such as methyl and ethyl radicals;
   R₁₆ is chosen from hydrogen and lower alkyl radicals such as methyl and ethyl radicals;
   R₁₇ is chosen from lower alkyl radicals, such as methyl and ethyl radicals, and radicals corresponding to the formula: -R₁₈-N(R₁₆)₂, wherein R₁₈ is chosen from —CH₂—CH₂—, —CH₂—CH₂—CH₂— and —CH₂—CH(CH₃)— groups, R₁₆ is as defined above, as well as the higher homologues of these radicals and those radicals containing up to 6 carbon atoms; and
   R₁₈ is defined as above.
(8) Amphoteric polymers of the type -D-X-D-X- chosen from:
   a) polymers obtained by the action of chloroacetic acid or sodium chloroacetate on compounds containing at least one unit of formula:
      -D-X-D-X-D- (XI)
   where D denotes a radical 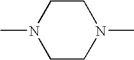
   and X is chosen from the symbols E and E′, wherein E and E′, which may be identical or different, are chosen from divalent radicals that are alkylene radicals with a straight or branched chain containing up to 7 carbon atoms in the main chain, which is optionally substituted with hydroxyl groups and that may contain, in addition to oxygen, nitrogen, and sulphur atoms, 1 to 3 aromatic and/or heterocyclic rings; the oxygen, nitrogen, and sulphur atoms being present in the form of ether, thioether, sulphoxide, sulphone, sulphonium, alkylamine, and alkenylamine group, hydroxyl, benzylamine, amine oxide, quaternary ammonium, amide, imide, alcohol, ester, and urethane groups;
   b) polymers of formula:
      -D-X-D-X- (XII)
   where D denotes a radical 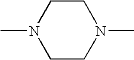
   and X is chosen from the symbols E and E′ and at least once E′; E having the meaning given above and E′ being a divalent radical that is an alkylene radical with a straight or branched chain having up to 7 carbon atoms in the main chain, which is optionally substituted with at least one hydroxyl radical and containing at least one nitrogen atom, the nitrogen atom being substituted with an alkyl chain that is optionally interrupted by an oxygen atom and contains at least one function chosen from carboxyl and hydroxyl functions and betainized by reaction with chloroacetic acid or sodium chloroacetate.
(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers may also contain other vinyl comonomers such as vinylcaprolactam.

The amphoteric polymers that may be mentioned are those of group (1), above.

The composition disclosed herein may contain at least one of the cationic or amphoteric polymers as described above.

According to certain embodiments disclosed herein, the at least one cationic or amphoteric polymer may be present in an amount ranging from 0.001% to 20% by weight, such as from 0.01% to 10% by weight or from 0.02% to 5% by weight, relative to the total weight of the final composition.

According to one embodiment, the compositions may also comprise at least one aminated silicone.

As used herein, the term “cosmetically acceptable medium” means a medium that is compatible with keratin materials, such as the skin, the eyelashes, and the hair.

The cosmetically acceptable medium may comprise only water or a mixture of water and at least one cosmetically acceptable solvent, such as C₁-C₄ lower alcohols, for instance ethanol, isopropanol, tert-butanol, n-butanol; alkylene glycols, for instance propylene glycol; and polyol ethers.

The composition may comprise from 50% to 95% by weight of water relative to the total weight of the composition.

The washing compositions disclosed herein may have a final pH ranging from 3 to 10. This pH may, for example, range from 5 to 8. The pH may be adjusted to the desired value conventionally, by adding a base (organic or mineral base) to the composition, for example aqueous ammonia or primary, secondary, or tertiary (poly)amines, for instance monoethanolamine, diethanolamine, triethanolamine, isopropanolamine, and 1,3-propanediamine, or alternatively by adding an acid, such as a carboxylic acid, for instance citric acid.

The compositions disclosed herein may contain, in addition to the combination defined above, viscosity regulators such as electrolytes and thickeners (associative and non-associative thickeners). Mention may be made of sodium chloride, sodium xylenesulphonate, scleroglucans, xanthan gums, fatty acid alkanolamides, alkyl ether carboxylic acid alkanolamides optionally oxyethylenated with up to 5 mol of ethylene oxide, such as the product sold under the name Aminol® A15 by the company Chem Y, crosslinked polyacrylic acids and acrylic acid copolymers such as crosslinked acrylic acid/C₁₀-C₃₀ alkyl acrylate copolymers. These viscosity regulators may be used in the compositions disclosed herein in an amount that may be up to 10% by weight relative to the total weight of the composition.

The compositions disclosed herein may also contain up to 5% of nacreous agents or opacifiers that are known in the art, such as, for example, fatty alcohols higher than C₁₆, fatty-chain acyl derivatives such as ethylene glycol polyethylene glycol monostearates, and polyethylene glycol distearates, and fatty-chain (C₁₀-C₃₀) ethers such as, for example, distearyl ether and 1-(hexadecyloxy)-2-octadecanol.

The compositions disclosed herein may optionally also comprise at least one additive chosen from foam synergists such as C₁₀-C₁₈ 1,2-alkanediols; fatty alkanolamides derived from monoethanolamine or diethanolamine; silicone or non-silicone sunscreens; anionic or nonionic polymers; cationic surfactants; proteins; protein hydrolysates; ceramides; pseudoceramides; linear or branched C₁₂-C₄₀ fatty acids such as 18-methyleicosanoic acid; hydroxy acids; vitamins; provitamins such as panthenol; animal, mineral, or synthetic oils; and any other additive conventionally used in cosmetics that does not affect the properties of the compositions disclosed herein.

The washing compositions disclosed herein may also comprise any adjuvant usually encountered in the field of shampoos, for instance fragrances, preserving agents, sequestering agents, softeners, dyes, moisturizers, anti-dandruff agents, anti-seborrhoeic agents, and the like.

A person skilled in the art will take care to select this or any optional additional compound(s) and/or the amounts thereof such that the advantageous properties intrinsically associated with the combination in accordance with the present disclosure are not, or are not substantially, adversely affected by the envisioned addition(s).

These compositions may be in the form of optionally thickened liquids, creams, or gels, and they may be suitable for washing, caring for, and/or styling the hair.

Further disclosed herein is a cosmetic process for treating keratin materials, comprising applying an effective amount of a composition as described above to the keratin materials, and rinsing after an optional leave-in time.

According to one embodiment, the composition may be used as a shampoo.

When the compositions disclosed herein are used as standard shampoos, they are simply applied to wet hair and the lather generated by massaging or friction with the hands, after which the composition is removed, after an optional action time, by rinsing with water, the operation possibly being repeated one or more times.

Concrete, but in no way limiting, examples illustrating certain embodiments disclosed herein follow.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result.

### EXAMPLE 1

The following shampoo composition was prepared:

| | |
|---|---|
| Sodium lauryl ether sulphate (C12/C14 at 70/30) at 2.2 mol | 12 g AM |
| of ethylene oxide at 26% AM | |
| (AM = active material) | |
| Polydimethylsiloxane with a viscosity of 60,000 cSt (Fluid | 1.5 g |
| DC60 000 cst from Dow Corning) | |
| Copolymer of acrylamide and dimethylaminoethyl | 0.8 g |
| quaternized with benzyl chloride sold under the name | |
| Ultimer ® by the company Ondeo | |
| Sodium chloride | 2 g |
| Citric acid or NaOH qs | pH 6.5 |
| Water qs | 100 g |

Hair treated with this shampoo had good styling and volumizing properties.

### EXAMPLES 2 to 5

| | 2 | 3 | 4 | 5 |
|---|---|---|---|---|
| Copolymer (90 mol/10 mol) of | 1% | 0.8% | — | 0.5% |
| acrylamide and of dimethyl- | AM | AM | | AM |
| aminoethyl acrylate quaternized | | | | |
| with benzyl chloride (MW > | | | | |
| 5 × 10⁶) as a dispersion in a | | | | |
| concentrated saline aqueous | | | | |
| solution (Ultimer ® from Ondeo) | | | | |
| Copolymer (10 mol/90 mol) of | — | — | 0.5% | — |
| dimethylaminoethyl acrylate | | | AM | |
| quaternized with methyl chloride | | | | |
| and of acrylamide (MW > 5 × 10⁶) | | | | |
| as a dispersion in a | | | | |
| concentrated saline aqueous | | | | |
| solution | | | | |
| Sodium lauryl ether sulphate | 12.5% | 12% | 7.5% | 10% |
| 2.2 EO | AM | AM | AM | AM |
| Cocoamidopropylbetaine | 2.5% | 1.5% | 2.5% | 2% |
| | AM | AM | AM | AM |
| Dimethicone | 1% | 1.5% | — | — |
| (DC 200 Fluid 60 000 cSt from | | | | |
| Dow Corning) | | | | |
| Dimethicone | — | — | 1% | — |
| (DC 200 Fluid 1000 cSt from | | | | |
| Dow Corning | | | | |
| Phenyl Trimethicone | — | — | — | 0.8% |
| (Belsil ® PDM 1000 from Wacker) | | | | |
| Ethanol | — | 1% | — | — |
| NaCl | — | — | — | 2% |
| Water qs | 100% | 100% | 100% | 100% |

Hair treated with these compositions had the same properties as hair treated with the composition of Example 1.

1. A composition for washing keratin materials comprising, in a cosmetically acceptable aqueous medium, at least one nonaminated silicone with a viscosity lower than 100,000 mm²/s (cSt), at least one detergent surfactant chosen from anionic, nonionic, and amphoteric detergent surfactants, and at least one drawing polymer with a drawing power of greater than 5 cm.

2. The composition according to claim 1, wherein the at least one detergent surfactant is chosen from anionic surfactants.

3. The composition according to claim 1, wherein the at least one detergent surfactant is present in an amount ranging from 4% to 50% by weight, relative to the total weight of the composition.

4. The composition according to claim 3, wherein the atleast one detergent surfactant is present in an amount ranging from 8% to 25% by weight, relative to the total weight of the composition.

5. The composition according to claim 1, wherein the drawing polymer is chosen from:
(a1) dispersions of particles of at least one water-soluble cationic polymer with a weight-average molecular mass of greater than 10⁶ in a saline aqueous solution, obtained by heterogeneous free-radical polymerization of water-soluble monomers with precipitation of the polymer formed, and (a2) aqueous solutions of at least one water-soluble cationic polymer with a weight-average molecular mass of greater than 10⁶, obtained by heterogeneous free-radical polymerization of water-soluble monomers with precipitation of the polymer formed.

6. The composition according to claim 5, wherein the at least one water-soluble monomer is chosen from at least one of cationic and nonionic monomers comprising at least one ethylenic double bond.

7. The composition according to claim 6, wherein the nonionic monomers are chosen from acrylamide, methacrylamide, N-vinylformamide, N-vinylacetonamide, hydroxypropyl acrylate, and hydroxypropyl methacrylate.

8. The composition according to claim 6, wherein the cationic monomers are chosen from di(C₁₋₄ alkyl)diallylammonium salts and compounds of formula (I) 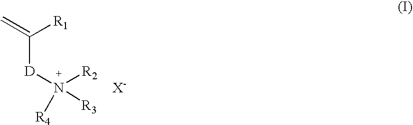
in which
R₁ is chosen from hydrogen and methyl groups,
R₂ and R₃, which may be identical or different, are each chosen from hydrogen and linear or branched C₁₋₄ alkyl groups,
R₄ is chosen from hydrogen, linear or branched C₁₋₄ alkyl groups, and aryl groups,
D represents the following unit 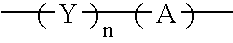
in which Y is chosen from amide, ester, urethane, and urea functions,
A is chosen from linear, branched, or cyclic C₁₋₁₀ alkylene groups, optionally substituted and optinally interrupted with at least one of divalent aromatic rings and heteroaromatic rings, and optionally interrupted with at least one hetero atom chosen from O, N, S, and P, and optionally comprising at least one ketone, amide, ester, urethane, and urea function,
n is chosen from 0 and 1, and
X⁻ represents an anionic counterion.

9. The composition according to claim 8, wherein the anionic counterion is chosen from halide and sulphate ions.

10. The composition according to claim 5, wherein the at least one water-soluble cationic polymer is polymerized using at least one cationic monomer of formula (I) 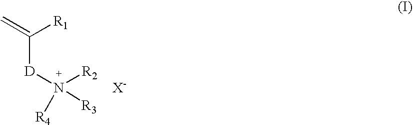
in which
R₁ is chosen from hydrogen and methyl groups,
R₂ and R₃, which may be identical or different, are each chosen from hydrogen and linear or branched C₁₋₄ alkyl groups,
R₄ is chosen from hydrogen, linear or branched C₁₋₄ alkyl groups, and aryl groups,
D represents the following unit 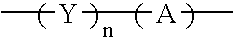
in which Y is chosen from amide, ester, urethane, and urea functions,
A is chosen from linear, branched, or cyclic C₁₋₁₀ alkylene groups, optionally substituted and optinally interrupted with at least one of divalent aromatic rings and heteroaromatic rings, and optionally interrupted with at least one hetero atom chosen from O, N, S, and P, and optionally comprising at least one ketone, amide, ester, urethane, and urea function,
n is chosen from 0 and 1, and
X⁻ represents an anionic counterion.

11. The composition according to claim 10, wherein the at least one water-soluble cationic polymer is polymerized using a monomer mixture comprising from 0 to 95.5 mol % of acrylamide and from 4.5 to 100 mol % of at least one cationic monomer of formula (I).

12. The composition according to claim 10, wherein the at least one water-soluble cationic polymer is polymerized using a monomer mixture comprising acrylamide and a cationic monomer of formula (I), the number of moles of the cationic monomer of formula (I) being greater than the number of moles of acrylamide.

13. The composition according to claim 10, wherein the at least one water-soluble cationic polymer is polymerized using a monomer mixture comprising 10 mol % of acryloyloxyethyldimethylbenzylammonium chloride and 90 mol % of acrylamide.

14. The composition according to claim 10, wherein the at least one water-soluble cationic polymer is polymerized using a monomer mixture comprising 30 mol % of acryloyloxytrimethylammonium chloride, 50 mol % of acryloyloxyethyldimethylbenzylammonium chloride, and 20 mol % of acrylamide.

15. The composition according to claim 10, wherein the at least one water-soluble cationic polymer is polymerized using 10 mol % of acryloyloxyethyltrimethylammonium chloride and 90 mol % of acrylamide.

16. The composition according to caim 10, wherein the at least one water-soluble cationic polymer is polymerized using 30 mol % of diallyldimethylammonium chloride and 70 mol % of acrylamide.

17. The composition according to claim 5, wherein the concentration of particles the at least one water-soluble cationic polymer as a dispersion in a saline aqueous solution in (a1) ranges from 0.01% to 20% by weight, relative to the total weight of the dispersion.

18. The composition according to claim 5, wherein the concentration of the at least one water-soluble polymer as a solution in (a2) ranges from 0.01% to 20% by weight, relative to the total weight of the solution.

19. The composition according to claim 5, wherein the aqueous solution in (a1) or (a2) comprises at least one anionic salt.

20. The composition according to claim 19, wherein the at least one anionic salt is chosen from ammonium sulphate, ammonium hydrogen sulphate, ammonium chloride, sodium sulphate, sodium hydrogen sulphate, magnesium sulphate, magnesium hydrogen sulphate, aluminium sulphate, and aluminium hydrogen sulphate.

21. The composition according to claim 1, wherein the at least one drawing polymer is present in the composition in an amount ranging from 0.01% to 10% by weight, relative to the total weight of the composition.

22. The composition according to claim 21, wherein the at least one drawing polymer is present in the composition in an amount ranging from 0.05% to 5% by weight, relative to the total weight of the composition.

23. The composition according to claim 1, wherein the at least one nonaminated silicone has a viscosity ranging from 1,000 to 100,000 mm²/s (cSt).

24. The composition according to claim 23, wherein the at least one nonaminated silicone has a viscosity ranging from 10,000 to 80,000 mm²/s (cSt).

25. The composition according to claim 1, wherein the at least one nonaminated silicone is a nonvolatile polyorganosiloxane chosen from at least one of polyalkylsiloxanes, polydialkylsiloxanes, polyarylsiloxanes, polyalkylarylsiloxanes, silicone resins, polyorganosiloxanes modified with at least one organofunctional group.

26. The composition according to claim 25, wherein the polydialkylsiloxanes are chosen from:
polydimethylsiloxanes containing trimethylsilyl end groups;
polydimethylsiloxanes containing dimethylsilanol end groups; and
poly(C₁-C₂₀)alkylsiloxanes.

27. The composition according to claim 25, wherein the polyalkylarylsiloxanes are chosen from:
polydimethylmethylphenylsiloxanes, and linear and/or branched polydimethyldiphenylsiloxanes with a viscosity ranging from 1×10⁻⁵ to 5×10⁻² m²/s at 25° C.

28. The composition according to claim 25, wherein the polyorganosiloxanes modified with at least one organofunctional group are chosen from silicones comprising in their structure at least one organofunctional group attached via a hydrocarbon-based radical.

29. The composition according to claim 25, wherein the polyorganosiloxanes modified with at least one organofunctional group are chosen from the polyorganosiloxanes comprising at least one group chosen from:
a) polyethyleneoxy groups;
b) thiol groups;
c) alkoxylated groups;
d) hydroxylated groups;
e) acyloxyalkyl groups;
f) alkylcarboxylic groups;
g) 2-hydroxyalkylsulphonate groups;
h) 2-hydroxyalkylthiosulphonate groups; and
i) polypropyleneoxy groups.

30. The composition according to claim 1, wherein the at least one nonaminated silicone is chosen from polydialkylsiloxanes containing trimethylsilyl end groups, polydialkylsiloxanes containing dimethylsilanol end groups, and polyalkylarylsiloxanes.

31. The composition according to claim 1, wherein the at least one nonaminated silicone is chosen from polyorganosiloxanes that are insoluble in the composition.

32. The composition according to claim 1, wherein the at least one nonaminated silicone is present in an amount ranging from 0.001% to 20% by weight, relative to the total weight of the composition.

33. The composition according to claim 32, wherein the at least one nonaminated silicone is present in an amount ranging from 0.01% to 10% by weight, relative to the total weight of the composition.

34. The composition according to claim 1, further comprising at least one cationic polymer.

35. The composition according to claim 1, further comprising at least one aminated silicone.

36. The composition according to claim 1, wherein the composition has a pH ranging from 3 to 10.

37. A method of using a cosmetic composition for cleaning, caring for, conditioning, and/or styling hair comprising applying the composition to hair, wherein said composition comprises, in a cosmetically acceptable aqueous medium, at least one nonaminated silicone with a viscosity lower than 100,000 mm²/s (cSt), at least one detergent surfactant chosen from anionic, nonionic, and amphoteric detergent surfactants, and at least one drawing polymer with a drawing power of greater than 5 cm.
